(19) United States
(12) Patent Application Publication
(10) Pub. No.: US 2025/0090754 A1
(43) Pub. Date: Mar. 20, 2025

(54) METHOD TO AVOID HYPOGLYCEMIA BY MINIMIZING LATE POST-PRANDIAL INSULIN INFUSION IN AID SYSTEM

(71) Applicant: University of Virginia Patent Foundation, Charlottesville, VA (US)

(72) Inventors: Marc D. BRETON, Charlottesville, VA (US); Jenny Lorena DIAZ CASTANEDA, Charlottesville, VA (US); Patricio H. COLMEGNA, Charlottesville, VA (US); Jose GARCIA-TIRADO, Bern (CH); Maria Fernanda VILLA TAMAYO, Charlottesville, VA (US); Marcela MOSCOSO-VASQUEZ, Charlottesville, VA (US)

(73) Assignee: University of Virginia Patent Foundation, Charlottesville, VA (US)

(21) Appl. No.: 18/887,538

(22) Filed: Sep. 17, 2024

Related U.S. Application Data
(60) Provisional application No. 63/583,371, filed on Sep. 18, 2023.

(51) Int. Cl. A61M 5/172; G16H 20/17

(52) U.S. Cl. CPC A61M 5/1723; G16H 20/17; A61M 2230/201

(57) ABSTRACT

Embodiments can relate to an insulin delivery controller which implements a processor configuration to efficiently attain an insulin delivery target. The insulin delivery controller can include a processor and a memory associated with the processor. The processor can process glucose data received from the memory, including a data representation of glycemic disturbance (d(t)). The processor can determine a glucose rate of change (G′(t)). The processor can generate a command signal to dynamically reshape a glycemic disturbance within a prediction horizon of the insulin delivery controller according to the G′(t). The processor can generate an insulin command signal for an insulin delivery unit to adjust an insulin delivery dosage amount and/or an insulin delivery dosage rate.

### CROSS-REFERENCE TO RELATED APPLICATIONS

This patent application is related to and claims the benefit of priority to U.S. Provisional Patent Application No. 63/583,371, filed on Sep. 18, 2023, the entire contents being incorporated herein by reference.

### FIELD

Embodiments can relate to an insulin delivery controller which implements a processor configuration to efficiently attain an insulin delivery target.

### BACKGROUND INFORMATION

The performance of model-based automatic insulin delivery (AID) systems can be dependent on how glycemic disturbances are modeled and projected over time. However, there is still no clear solution to this problem due to the high uncertainty around glucose dynamics.

### SUMMARY

An exemplary embodiment can relate to an insulin delivery controller which implements a processor configuration to efficiently attain an insulin delivery target. The insulin delivery controller can include a processor and a memory associated with the processor. The memory can include instructions stored thereon that when executed by the processor will cause the processor to perform one or more functions described herein. Instructions can cause the processor to process glucose data received from the memory, including a data representation of glycemic disturbance (d(t)). Instructions can cause the processor to determine a glucose rate of change (G′(t)). Instructions can cause the processor to generate a command signal to dynamically reshape a glycemic disturbance within a prediction horizon of the insulin delivery controller according to the G′(t). Instructions can cause the processor to generate an insulin command signal for an insulin delivery unit to adjust an insulin delivery dosage amount and/or an insulin delivery dosage rate.

An exemplary embodiment can relate to a computer readable medium including instructions stored thereon that when executed by a processor will cause the processor to efficiently attain an insulin delivery target. Instructions can cause the processor to efficiently attain an insulin delivery target by processing glucose data including a data representation of glycemic disturbance (d(t)). Instructions can cause the processor to efficiently attain an insulin delivery target by determining a glucose rate of change (G′(t)). Instructions can cause the processor to efficiently attain an insulin delivery target by generating a command signal to dynamically reshape a glycemic disturbance within a prediction horizon of an insulin delivery controller according to the G′(t). Instructions can cause the processor to efficiently attain an insulin delivery target by generating an insulin command signal for an insulin delivery unit to adjust an insulin delivery dosage amount and/or an insulin delivery dosage rate.

An exemplary embodiment can relate to a method for managing a processor configuration to efficiently attain an insulin delivery target. The method can involve processing glucose data including a data representation of glycemic disturbance (d(t)). The method can involve determining a glucose rate of change (G′(t)). The method can involve generating a command signal to dynamically reshape a glycemic disturbance within a prediction horizon of an insulin delivery controller according to the G′(t). The method can involve generating an insulin command signal for an insulin delivery unit to adjust an insulin delivery dosage amount and/or an insulin delivery dosage rate.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1 shows an exemplary insulin delivery controller architecture;

FIG. 2 shows an exemplary insulin control system architecture;

FIG. 3 is an example of d-shaping along the prediction horizon based on the rate of change of glucose;

FIG. 4 shows a timeline of BPS comparison sessions and challenge days;

FIG. 5 shows change in glucose levels (top) and percentage of insulin infused over 3 h (bottom) over time after a meal for the AID system with and without BPS engaged;

FIG. 6 shows percentage of insulin infused over the first 3 h after a meal for the AID system with and without BPS engaged;

FIG. 7A shows a closed-loop response for 100 in-silico individuals with and without the disturbance shaping feature: Solid line—median values; Dark shaded area—25-75^{th} percentiles; Light shaded area—5-95^{th} percentiles;

FIG. 7B shows distributions of 15-g hypoglycemia rescue carbohydrates used in-silico with and without disturbance shaping;

FIG. 8 is an exemplary high-level functional block diagram for an embodiment of an insulin delivery controller;

FIG. 9 shows an exemplary computing device configuration;

FIG. 10 is a block diagram that illustrates an exemplary system including a computer system and an associated Internet connection upon which an embodiment of the insulin delivery controller may be implemented;

FIG. 11 illustrates a system in which one or more embodiments of the insulin delivery controller can be implemented using a network, or portions of a network or computers; and

FIG. 12 is a block diagram illustrating an example of a machine upon which one or more aspects of embodiments of the insulin delivery controller can be implemented.

### DETAILED DESCRIPTION

Embodiments disclosed herein improve operation of one or more processors 102. This can be achieved by implementing a model or algorithm that will improve efficiency of processor 102 operation by requiring less iterations, requiring less computational resources, etc. For instance, the model or algorithm can allow the processor 102 to stabilize operation more quickly so as to more efficiently attain an insulin delivery target. Known systems and methods overshoot or undershot the target and then (in some cases) overshoot or undershoot again via an over-correction, etc. Via implementation of embodiments of the model or algorithm disclosed herein, the processor 102 can more quickly and efficiently attain a delivery target, thereby providing higher precision (e.g., the selected dosage is closer to the target), requiring less computational resources (e.g., requiring less iterations, requiring less processor size and components, etc.), etc.

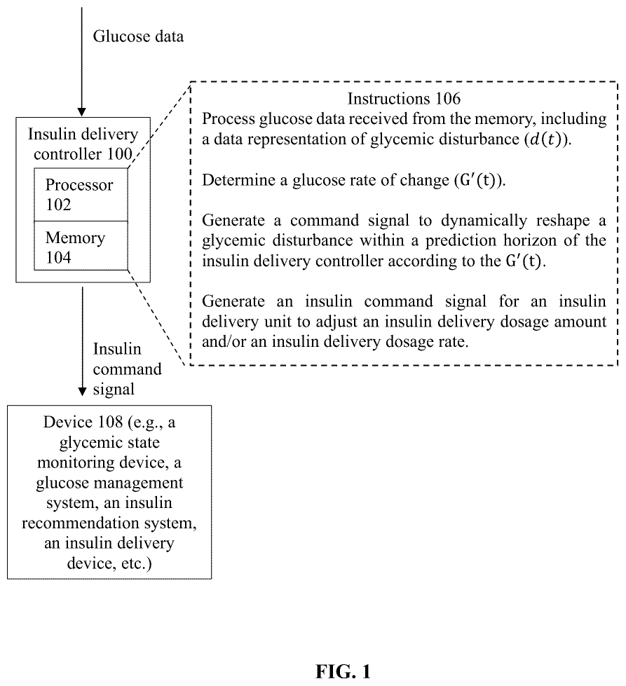

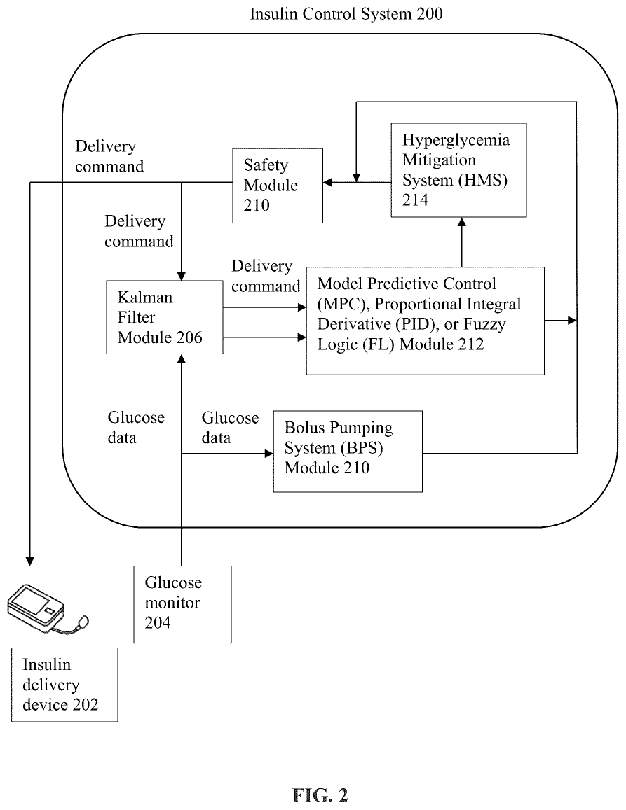

Referring to FIGS. 1-2, embodiments can relate to an insulin delivery controller 100 which can implement a processor 102 configuration to efficiently attain an insulin delivery target. The insulin delivery controller 100 can include a processor 102. The insulin delivery controller 100 can include a memory 104. The memory 104 may be associated with the processor 102.

The processor 102 can be any of the processors 102 disclosed herein. The processor 102 can be part of or in communication with a machine (logic, one or more components, circuits (e.g., modules), or mechanisms). The processor 102 can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors 102 herein can include any one or combination of a Graphics Processing Unit (GPU), a Field Programmable Gate Array (FPGA), a Central Processing Unit (CPU), etc. The processor 102 can include one or more processing modules. A processing module can be a software or firmware operating module configured to implement any of the method steps disclosed herein. The processing module can be embodied as software and stored in memory 104, the memory 104 being operatively associated with the processor 102. A processing module can be embodied as a web application, a desktop application, a console application, etc. Exemplary embodiments of the processor 102 and the machine are discussed later.

The processor 102 can include or be associated with a computer or machine readable medium. As discussed in more detail later, the computer or machine readable medium can include memory 104. The computer or machine readable medium can be configured to store one or more instructions 106 thereon. The instructions 106 can be in the form of algorithms, program logic, etc. that cause the processor 102 to build and implement embodiment of the model.

Any of the memory 104 discussed herein can be computer readable memory configured to store data. The memory 104 can include a volatile or non-volatile, transitory or non-transitory memory, and be embodied as an in-memory, an active memory, a cloud memory, etc. Embodiments of the memory 104 can include a processor module and other circuitry to allow for the transfer of data to and from the memory 104, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The processor 102 can be in communication with other processors of other devices 108 (e.g., a glycemic state monitoring device, a glucose management system, an insulin recommendation system, an insulin delivery device, a non-portable computer device, a portable computer device, a hand-held computer device, a wearable computer device, a smartphone, a smartwatch, etc.). Any of those other devices 108 can include any of the exemplary processors disclosed herein. Any of the processors can have transceivers or other communication devices/circuitry to facilitate transmission and reception of wireless signals. Any of the processors can include an Application Programming Interface (API) as a software intermediary that allows two applications to talk to each other. Use of an API can allow software of the processor 102 of the system 100 to communicate with software of the processor of the other device(s) 108, if the processor 102 of the system 100 is not the same processor of the device 108.

Any data transmission between the processor 102 and memory 104, between the processor 102 and a database, and between the processor 102 and processors of other devices 108, etc. can be via a pull operation (e.g., the processor 102 can pull the data) or a push operation (e.g., the data can be pushed to the processor 102). The processor 102 can receive the data in steaming format, or store it in memory 104 before being processed. In addition, embodiments of the model disclosed herein can be developed an application software (an “App”) to be implemented on a processor of a device 108 (e.g., a wearable device). The App can be sent via a steaming format, or the App can be sent and stored on a memory associated with or accessed by the device 108.

As noted herein, the processor 102 can be configured to be a component of, used in combination with, or in communication with another device/system 100—e.g., this can include the processor 102 being part of the device/system 100, the device/system 100 being part of the processor 102, the processor 102 in communication with the device/system 100, etc. “Being part of” can include being on a same substrate or integrated circuit. For instance, the processor 102 can be a component of, used in combination with, or in communication with a predictive modeling system (e.g., a system for predicting risk of hypo- or hyper-glycemia), a decision support system (e.g., a system for assisting with medical triage), an automated control system (e.g., an artificial pancreas), etc. The processor 102 can use the model or algorithm or provide the model or algorithm to the device/system to assist with or augment the performance of these devices/systems. Output of the model or algorithm can be used by the device/system to assist with or augment predicting or reacting to aspects of glycemic states, assist with or augment determining or modifying insulin administration therapies, etc.

As noted herein, the processor 102 can be configured to be a component of, used in combination with, or in communication with a predictive modeling system, a decision support system, an automated control system, a non-portable computer device, a portable computer device, a hand-held computer device, a wearable computer device, an insulin monitoring device, an insulin delivery device, etc. For instance, the processor 102 can be configured to be a component of, used in combination with, or in communication with an insulin delivery device 108, wherein the instructions 106 can cause the processor 102 to generate a command signal configured to cause the insulin delivery device 108 to adjust an insulin delivery dosage amount and/or an insulin delivery dosage rate.

As can be appreciated from the disclosure, the memory 104 can include instructions 106 stored thereon that when executed by the processor 102 will cause the processor 102 to perform one or more functions described herein. For instance, instructions 106 can cause the processor 102 to process glucose data received from the memory 104. The glucose data can be a measure(s) of blood glucose, interstitial fluid glucose, etc. The glucose data can be measured by a glucose sensor, glucose meter, glucose monitor, etc. The glucose data can be transmitted to the memory 104 in real time, periodically, as requested, continuously, in batches, sent directly to the memory 104 sent to a different data store (e.g., database) before being transmitted to the memory 104, etc. The glucose data can include a data representation of glycemic disturbance (d(t)). A disturbance is something (e.g., an event such as a meal, exercise, stress, etc.) that causes a subject's glycemic state to deviate away from a target glycemic state. This can include deviating from a target glycemic state in a closed loop implementation. Thus, a disturbance can be represented in the glucose data as a glycemic measurement (e.g., glucose data) that is an excursion from a predicted, expected, desired, etc. glycemic measurement. The disturbance can be represented in the glucose data as plural glycemic measurements that are trending from a predicted, expected, or desired set of glycemic measurements. The disturbance can represented in the glucose data as a predicted output of glycemic measurement(s) that are deviating, trending, expected to deviate, expected to trend, etc. from a desired glycemic measurement(s). Similarly, a disturbance can be represented in the glucose data as a glycemic data profile(s) (e.g., plural data points pertaining to a glycemic state) that deviate, are predicted to deviate, etc. from an expected glycemic data profile(s). The deviation or excursion can be measured as being beyond a threshold, extending beyond a central tendency, having a threshold number of data points extending beyond an upper or lower bound, having a threshold number of data points extending beyond an upper or lower bound for a predetermine range of time, etc. The disturbance itself can be an actual excursion value, deviation value, an estimate, a prediction, etc. The disturbance can be defined as a function—e.g., a function of time. For instance, the disturbance can be glucose measurement data as a function of time (e.g., a time series of plural glucose measurements) that include one or more excursion or deviation values. The disturbance can be defined as (\hat{d}(t)), for example.

Instructions 106 can cause the processor 102 to determine a glucose rate of change (G′(t)). This can be a projection of (d(t)) based on a current (\hat{d}(t)) (e.g., most recent data representative of a disturbance, most recent data collected, data that falls within a predetermine time window, etc.), a current estimate of (\hat{d}(t)), etc. This projection can be a projection of (\hat{d}(t)) within a prediction horizon. A prediction horizon can refer to the number of future control intervals a control system must evaluate by prediction when optimizing at a set control interval. With a model predictive control (MPC) technique, for example, the prediction horizon can be an amount of time into the future that a MPC algorithm predicts a system's behavior within a predetermine level of accuracy or precision when deciding on one or more control inputs.

As a non-limiting example, (G′(t)) can be as follows:
if G′(t)>G′_{Lim}
   d(t) will be constant over the prediction horizon at the estimated value \hat{d}(t), being
   G′_{Lim} the limit change in the glucose level to modify d(t) along the prediction horizon.
else
   d(t) will decrease from \hat{d}(t) to \hat{d}(t)—d*(t) at the end of the prediction horizon, with d*(t) computed as follows:

$${d}^{*} ( t ) = \frac{{\%}_{ROC - {G}^{′}}}{100} * \hat{d} ( t )$$

with %_{ROC-G′} the percentage of the glucose rate of change for a maximum allowable change in the glucose level (G̅′̅) computed as follows:

$${\%}_{ROC - {G}^{′}} = min ( 100 , ( \frac{100 * \vert {G}^{′} ( t ) \vert}{\overline{{G}^{′}}} ) ) .$$

Instructions 106 can cause the processor 102 to generate a command signal to dynamically reshape a glycemic disturbance within a prediction horizon of the insulin delivery controller. This command signal can be based on, as a function of, in accordance with, etc. G′(t). For instance, when G′(t)≤G′_{Lim}, the rate at which d(t) decreases over the prediction horizon can be modulated as a function of G′(t). With this exemplary modulation scheme, when |G′(t)|≥G̅′̅, d(t) decreases from its current estimated value \hat{d}(t) until zero at the end of the prediction horizon. Both G̅′̅ and G′_{Lim} are tunable parameters that can be used to adjust the insulin delivery controller 100 aggressiveness.

Instructions can cause the processor 102 to generate an insulin command signal for an insulin delivery unit 108. This command signal can be a signal that requires, suggests, recommends, etc. to adjust an insulin delivery dosage amount and/or an insulin delivery dosage rate of the insulin delivery unit 108. This adjustment can be based on, as a function of, in accordance with, etc. the modulation. It should be noted that the insulin command signal can be based on the modulation, wherein the modulation can be a non-modulation—e.g., no adjustment. For instance, the prediction(s) of the disturbance can be a “no disturbance” or a disturbance but an acceptable level of disturbance that does not require adjustment, etc. In this scenario, the insulin command signal can require, suggest, recommend, no adjustment.

Instructions 106 can cause the processor 102 to generate the insulin command signal for an insulin delivery unit 108 that adjusts the insulin delivery dosage amount and/or the insulin delivery dosage rate to modulate insulin delivery. Insulin delivery can be modulated to minimize a risk of or prevent hypoglycemia, attenuate glycemic disturbances tending towards hyperglycemia, etc. This is in contrast to keeping a disturbance constant along the prediction horizon. Keeping the disturbance constant can lead to control actions that are too conservative at the beginning of the postprandial period and too aggressive at the end of it where glucose is likely to be falling. With systems that keep the disturbance constant, the processor 102 is not efficiency or accurately attaining a delivery target. Instead, the processor 102 has to administer a less-accurate insulin dosage, perform additional data processing, readjust an insulin dosage because of the too conservative or too aggressive control actions, and administer another insulin dosage to compensate, which results in inaccuracy in the system, less precision in the system, unnecessary expense of computational resources, increased processing power, increased memory, etc. Further, existing systems that that keep the disturbance constant might require additional safety modules to compensate for the lack of accuracy and precision.

FIG. 2 shows an exemplary insulin control system architecture that may be implemented as a closed loop control (“CLC”) in an insulin management system 200. The insulin management system 200 can be or include an insulin monitoring unit, and insulin delivery unit 202, a glycemic monitoring unit 204, etc. A CLC system includes one or more control algorithms for glucose regulation, which can be model predictive control (MPC), proportional integral derivative (PID), and fuzzy logic (FL), etc. A MPC algorithm can a predict future glucose level(s)/concentration(s) to bring current glucose level(s)/concentration(s) into the target range. A PID algorithm can analyze a deviation of a measured glucose level(s)/concentration(s) from a target range to calculate the amount of insulin to deliver. A fuzzy algorithm can mimic an insulin dose calculation(s) made by clinical experts based on monitoring data. One of the goals of such an algorithm can be to keep glucose level(s).concentration(s) in a safe range (e.g., not extend into, cause, have a tendency to cause, etc. a hypo- or hyper-glycemic state). The CLC system can integrate one or more of algorithms to modulate control strength of insulin infusion, for example. The CLC, in addition to the disturbance techniques disclosed herein, can be dependent on past control actions, glucose measurements, any derivatives thereof, etc.

In addition, or in the alternative, embodiments can be used to optimize CLC of an insulin management system. This can include optimization to automate or assist with automating insulin delivery. For instance, the insulin command signal can adjust the insulin delivery dosage amount and/or the insulin delivery dosage rate to modulate insulin delivery so that blousing is automated, as appropriate. As appropriate can include automatically modulating insulin delivery to reject, attenuate, dampen, increase, smoot out, etc. glycemic disturbances. This can be done to mitigate effects of glycemic disturbances tending toward hyperglycemia while inhibiting potential for hypoglycemia, for example.

As a non-limiting example, an insulin management system 200 can include a processor configured to implement a MPC. The system can include an embodiment of the insulin delivery controller 100 or be in communication therewith. The insulin management system can be implemented in a Diabetes Assistant (DiA) format provided by a smartphone, for example. The glucose meter 204 can be a Continuous Glucose Monitor (CGM), for example. The DiA may define a general control paradigm that predicts future glycemia values and calculates optimal insulin doses to maintain an individual's target glucose level/concentration. For instance, the DiAc can be configured to predict glycemia values and calculate insulin dosages based on a variation of any of the models disclosed herein. Based on any detected disturbance, the MPC (with its embedded the insulin delivery controller 100) can command an insulin pump 202, for example, to deliver basal insulin dosing (e.g., microboluses). The dosing can be modulated by modulating the aggressiveness or amount of such dosing as a function of the rate of glucose change. In this way, the MPC can optimized relative to a predetermined and fixed target glucose level such that hypoglycemic and hyperglycemic events may be substantially avoided More specifically, the MPC can request deviation from a fixed basal rate so as to yield optimal dosing.

The embodiment of the insulin management system 200 can include additional operating modules to assist with insulin control. These can include one or more of a Kalman filter module 206 (storing and/or implementing one or more algorithms or models to optimally estimate one or more variables of interests), a safety module 208 (storing and/or implementing one or more redundant algorithmic or model controls to avoid infusion of insulin that would create a hypo—and/or hyper-glycemic state), a bolus pumping system module 210 (storing and/or implementing one or more algorithms or models to generate an infusion of basal based on a basal rate), a MPC module 212 (storing and/or implementing one or more MPC control algorithms or models, including one or more disturbance shaping algorithms or models disclosed here), and a hyperglycemia mitigation system module 214 (storing and/or implementing algorithmic or model controls that take corrective action in the event a hyperglycemic state is detected). With the exemplary insulin management system 200, the glucose monitor 204 can feed glucose data to the Kalman filter module 206 in parallel with feeding glucose data to the bolus pumping system module 210. The glucose data can be processed by the Kalman filter module 206 and transmitted to the MPC module 212. Output from the MPC module 212 can be combined with output from the bolus pumping system module 210 via a first path. Output from the MPC module 212 can be transmitted to the hyperglycemia mitigation system module 214 via a second path. The output of the first path and output from the hyperglycemia mitigation system module 214 can be combined before being transmitted to the safety module 210. The safety module 210 can generate a delivery command that can be fed back through the system 200 via the Kalman filter module 206 and parallelly fed to an insulin delivery device 202.

As can be appreciated, the predicted glucose rate of change (G′(t)) can be adjusted via modulation of disturbance (\hat{d}(t)) within the prediction horizon. This modulation can be on a continual basis, periodic basis, as thresholds are met or passed, as certain deviations or detected, etc. Hence, the glycemic disturbance can be dynamically reshaped within the prediction horizon according to the glucose rate of change. As noted herein, instructions 106 can cause the processor 102 to estimate d(t), which can be done periodically (e.g., every five minutes). d(t) can be estimated using a Kalman filtering technique, for example. Instructions 106 can cause the processor 102 to hold d(t) constant or decrease it over the prediction horizon based on G′_{Lim}. For instance, when G′(t)>G′_{Lim}, instructions 106 can cause the processor 102 to generate a command signal for the insulin delivery controller 100 to hold d(t) constant. When G′(t)≤G′_{Lim}, instructions 106 can cause the processor 102 to generate a command signal for the insulin delivery controller 100 to decrease d(t) over the prediction horizon. When the instructions 106 cause the processor 102 to decrease d(t), G̅′̅ can be used to control the decrease. For instance, when |G′(t)|≥G̅′̅, instructions 106 can cause the processor 1002 to generate a command signal for the insulin delivery controller 100 to decrease d(t) from its current estimated value \hat{d}(t) to zero at the end of the prediction horizon, and when |G′(t)|>G̅′̅, instructions 106 can cause the processor 102 to generate a command signal for the insulin delivery controller 100 to decrease \hat{d}(t) from \hat{d}(t) to \hat{d}(t)−d*(t) with d*(t) computed as a function of G′(t).

As noted herein, instructions 106 can cause the processor 102 to process the glucose data, determine the (G′(t)), and generate the command signal to dynamically reshape the glycemic disturbance via a closed loop control (CLC) process. For instance, the processor 102 can implement, as part of the CLC process, a model predictive control (MPC) algorithm, a proportional integral derivative (PID) algorithm, a fuzzy logic (FL) algorithm, etc.

In some embodiments, instructions 106 can cause the processor 102 to generate the insulin command signal for an insulin delivery unit 108 that adjusts the insulin delivery dosage amount and/or the insulin delivery dosage rate to modulate insulin delivery. Insulin delivery can be modulated to approach a target glucose level.

Some embodiments can include the insulin delivery controller 100 in combination with an insulin delivery unit 108. The insulin delivery unit 108 can include a processor and a memory with instructions stored thereon that when executed by the insulin delivery processor will cause the insulin delivery processor to generate an insulin delivery dose based on the insulin delivery dosage amount and/or the insulin delivery dosage rate.

An exemplary embodiment can relate to a computer readable medium including instructions 106 stored thereon that when executed by a processor 102 can cause the processor 102 to efficiently attain an insulin delivery target. Instructions 106 can cause the processor 102 to efficiently attain an insulin delivery target by processing glucose data including a data representation of glycemic disturbance (d(t)). Instructions 106 can cause the processor 102 to efficiently attain an insulin delivery target by determining a glucose rate of change (G′(t)). Instructions 106 can cause the processor 102 to efficiently attain an insulin delivery target by generating a command signal to dynamically reshape a glycemic disturbance within a prediction horizon of an insulin delivery controller 100 according to the G′(t). Instructions 106 can cause the processor 102 to efficiently attain an insulin delivery target by generating an insulin command signal for an insulin delivery unit 108 to adjust an insulin delivery dosage amount and/or an insulin delivery dosage rate.

An exemplary embodiment can relate to a method for managing a processor configuration to efficiently attain an insulin delivery target. The method can involve processing glucose data including a data representation of glycemic disturbance (d(t)). The method can involve determining a glucose rate of change (G′(t)). The method can involve generating a command signal to dynamically reshape a glycemic disturbance within a prediction horizon of an insulin delivery controller 100 according to the G′(t). The method can involve generating an insulin command signal for an insulin delivery unit 108 to adjust an insulin delivery dosage amount and/or an insulin delivery dosage rate.

### EXAMPLES

The following are exemplary systems, methods, and implementations of the embodiments disclosed herein. While the examples may focus on one implementation, it is understood that this is exemplary and the embodiments disclosed herein are not limited thereto.

### Example I

As noted herein, the performance of model-based automatic insulin delivery (AID) systems is highly dependent on how glycemic disturbances are modeled and projected over time. Note that how a disturbance is projected over time has a direct impact on glucose predictions and how the controller will proactively regulate the insulin infusion. This is particularly critical during a postprandial period, because overestimated disturbances can lead to late hypoglycemia. For example, keeping a disturbance constant along the prediction horizon could lead to control actions that are too conservative at the beginning of the postprandial period and too aggressive at the end of it where glucose is likely to be falling. In this regard, embodiments disclosed herein can dynamically reshape glycemic disturbances within the controller's prediction horizon according to the glucose rate of change.

Let us consider a glycemic disturbance denoted as d(t) that is estimated every five minutes, for example, using a Kalman filter. Then, based on the current estimation of the disturbance (\hat{d}(t)), a function is proposed to project it based on the glucose rate of change (G′(t)) as follows:
if G′(t)>G′_{Lim}
   d(t) will be constant over the prediction horizon at the estimated value \hat{d}(t), being G′_{Lim} the limit change in the glucose level to modify d(t) along the prediction horizon.
else
   d(t) will decrease from \hat{d}(t) to \hat{d}(t)—d*(t) at the end of the prediction horizon, with d*(t) computed as follows:

$${d}^{*} ( t ) = \frac{{\%}_{ROC - {G}^{′}}}{100} * \hat{d} ( t )$$

with %_{ROC-G′} the percentage of the glucose rate of change for a maximum allowable change in the glucose level (G̅′̅) computed as follows:

$${\%}_{ROC - {G}^{′}} = min ( 100 , ( \frac{100 * \vert {G}^{′} ( t ) \vert}{\overline{{G}^{′}}} ) ) .$$

Note that, when G′(t)≤G′_{Lim}, the rate at which d(t) decreases over the prediction horizon is modulated as a function of G′(t). When |G′(t)|≥G̅′̅, d(t) decreases from its current estimated value \hat{d}(t) until zero at the end of the prediction horizon. Both G̅′̅ and G′_{Lim} are tunable parameters that can be used to adjust the controller's aggressiveness.

Case study:

In the context of our MPC-based AID system, consider the following mathematical model:

$$\dot{G} = - {S}_{g} ( G - {G}_{b} ) - ( Δ X + {X}_{o l} ) {S}_{I} G + d ( t ) \qquad 1 )$$

$$\dot{d} = 0 \qquad 2 )$$

$$Δ \dot{X} = - \frac{1}{1000} Δ X \qquad 3 )$$

$${\dot{X}}_{o l} = - {p}_{2} {X}_{o l} + {p}_{2} ( I - {I}_{b} ) \qquad 4 )$$

$${\dot{I}}_{sc 1} = - ( {k}_{1} + {k}_{d} ) {I}_{sc 1} + J ( t ) \qquad 5 )$$

$${\dot{I}}_{sc 2} = - {k}_{2} {I}_{sc 2} + {k}_{d} {I}_{sc 1} \qquad 6 )$$

$$\dot{I} = - nI + \frac{I {R}_{a}}{( {V}_{I} \cdot BW )} \qquad 7 )$$

$$with$$

$$\begin{matrix}I {R}_{a} = {k}_{1} {I}_{sc 1} + {k}_{2} {I}_{sc 2} \\ Δ X = {X}_{cl} - {X}_{ol}\end{matrix}$$

X_{cl} and Xₒₗ represent the filtered (after the Kalman filter) and non-filtered (open loop) insulin concentrations in the remote compartment, respectively. The model parameters and variables are reported in Tables 1 and 2.

To keep a manageable computational burden, the controller is design in the linear framework, meaning a linearization is performed around a subject-specific operating point, e.g., we set Gₚ=120 mg/dL, Jctrl=u_{b} with u_{b}=TDI/48 the basal insulin, TDI computed using the patient-specific insulin record, and then look for the steady-state solution of the modelError! Reference source not found. Then, a discretization is performed over the linearized system using a sampling time of 5 minutes and a zero-order-hold on the inputs.

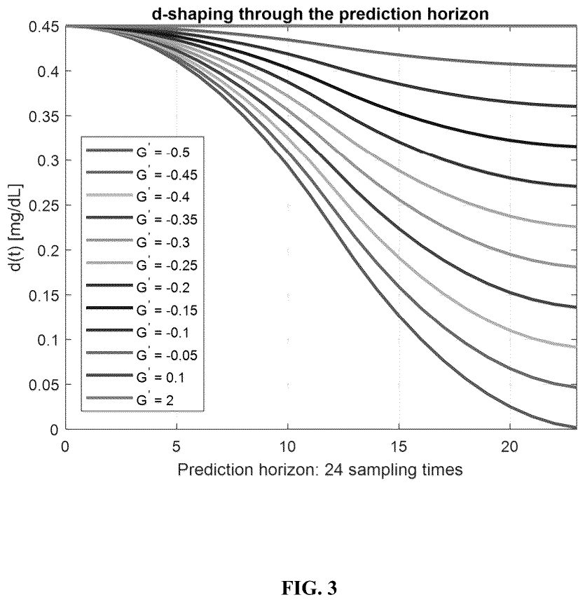

As mentioned above, since there is high uncertainty around d(t), it is modeled as an integrator and its value is estimated every five minutes using a Kalman filter. Then, an example of the d-shaping throughout the prediction horizon is presented in FIG. 3, assuming G′_{Lim}=0.05 mg/dL/min, \hat{d}(t)=0.45 mg/dL at time instant t, G̅′̅=0.5 mg/dL/min, and considering different values for G′.

As shown in FIG. 3, d(t) follows changes in G′(t) in order to inform the controller whether positive perturbations are expected to affect glucose levels within the prediction horizon. In this way, the controller's aggressiveness can be better modulated, and insulin infusion can be proactively reduced when glucose starts to decrease.

TABLE 1
Parameters with their respective units and values (population)

| | Nominal | |
|---|---|---|
| Symbol | Value | Units |
| S_{g} | 0.0046 | min⁻¹ |
| S_{I} | 6.62 × 10⁻⁴ | 1/min per mU/L |
| p₂ | 0.033 | min⁻¹ |
| k₁ | 0.001 | min⁻¹ |
| k_{d} | 0.018 | min⁻¹ |
| k₂ | 0.0132 | min⁻¹ |
| n | 0.127 | min⁻¹ |
| V_{I} | 9.31 | L |
| G_{b} | 120 | mg/dL |
| BW | User's | kg |
| | specific | |

TABLE 2
State variables with their corresponding units.

| Symbol | Meaning | Units |
|---|---|---|
| I_{sc1} | Amount of insulin, first compartment. | mU |
| I_{sc2} | Amount of insulin, second | mU |
| | compartment. | |
| X | Insulin concentration in the remote | mU/L |
| | compartment | |
| G | Blood glucose concentration | mg/dL |
| I | Insulin concentration in plasma | mU/L |
| J(t) | Exogenous insulin infusion rate | mU/min |
| d(t) | Disturbance signal | mg/dL |

### Example II

Automated insulin delivery (AID) is widely available to people with type 1 diabetes (T1D), providing superior glycemic control vs. traditional methods. The next generation of AID devices focus on minimizing user/device interactions, especially around meals (“full closed loop,” FCL). Our goal was to assess the postprandial glycemic impact of the bolus priming system (BPS), an algorithm delivering fixed insulin doses based on the likelihood of a meal having occurred, in conjunction with UVA's latest AID.

Eleven adults with T1D participated in a supervised randomized-crossover trial assessing two 24-hour sessions with identical meals and activity—with and without BPS. On the day in-between study sessions, participants underwent food and activity challenges to test BPS safety and robustness. Continuous glucose monitor (CGM) outcomes and total insulin doses were assessed overall and following meals with potential for BPS to dose additional insulin (CGM>90 mg/dL for one-hour prior).

Daytime CGM-outcomes were similar with and without BPS: time-in-range 70-180 mg/dL (TIR) 70.6% [62.2-76.5]vs 65.7% [58.6%-80.6%]; time-below-range<70 mg/dL (TBR) 0% [0-2.1]vs 0% [0-1.3]; respectively. Insulin delivery during 3 hours postprandial was almost identical 33.5 units [26.4-47.0]vs 35.7 [28.7-44.9]. Among 43 out of 66 meals with potential to trigger BPS (24/19 BPS/no-BPS), postprandial incremental area-under-the-curve (iAUC) was lower for BPS vs. No-BPS (2530±1934 vs 3228±2029, p<0.047), but CGM outcomes were inconclusive: 4 hour-TIR 51.2% [19.8-83.3]vs 40.2% [20.8-56.3](p=0.24). There were no severe adverse events.

When active (e.g., not following low glucose) BPS improved postprandial control in FCL via earlier insulin injection.

### Introduction

Glycemic control of Type 1 diabetes (T1D) has been substantially improved with use of automated insulin delivery (AID) systems, in which glycemia data from continuous glucose monitors (CGM) feed a computer algorithm to guide insulin delivery. AID systems improve glycemic time-in-range 70-180 mg/dL by 9-15% compared to prior care. Currently-available AID systems are designed to be primarily used in hybrid closed-loop control (HCL), e.g., still requiring users to enter data on ingested carbohydrate. However, persons with T1D commonly omit boluses for food ingestion and research has focused on the feasibility of systems that minimize (or altogether cancel) the need for meal announcement, a use modality also referred to as full closed loop or FCL.

Maintaining adequate insulin delivery around meals poses the most difficult challenge to maintaining glycemic control for AID systems used as FCL, because in most cases carbohydrates are absorbed more quickly than insulin. We previously reported a model-predictive controller (MPC) AID system that included a feature called the “bolus priming system” (BPS), which instructs the controller to deliver a rapid insulin dose after detecting a disturbance in glucose levels that the system interprets as having a high probability of representing a recent meal ingestion. When used as FCL, this system provided superior TIR compared to a commercially-available system. However, we had not previously determined if the BPS feature itself offered improved glycemic control in the absence of meal announcement compared to the MPC system with the BPS module silenced. Also, because these BPS doses were triggered by glucose disturbances that were not always related to food ingestion, we also realized there may be risks to such a system—for example, if an abrupt rise in glucose was triggered by intense anaerobic activity, such as from high-intensity interval training (HIIT) or ingestion of a small amount of rapidly-absorbed glucose. We also wondered about the safety of such a system during delayed gastric emptying, such as following ingestion of a high-carbohydrate, high-fat meal like pizza.

Our goals in the current project were to (i) compare this system as FCL with and without BPS, for the daytime period and in the time following meals and (ii) to assess the safety of such a system in a series of glycemic challenges. We hypothesized that we would observe improved glycemic control with vs. without BPS. These data have significance for incorporation of bolus priming in developing AID systems used as FCL.

### Research Design and Methods

The University of Virginia Institutional Review Board (IRB-HSR #22026) and the Food & Drug Administration (IDE #G220204) approved this randomized controlled clinical trial (ClinicalTrials.gov NCT05528770). Each participant provided written informed consent. Inclusion criteria included age 18-65 years, a documented diagnosis of T1D and insulin pump therapy for ≥3 months. Exclusion criteria included diabetic ketoacidosis (DKA) or a severe hypoglycemic event (defined as seizure or loss of consciousness) in the past 6 months, use of an oral glucose-lowering agent including metformin, pregnancy, and any medical condition deemed high-risk by the clinical investigators.

Enrollment and screening visits were performed via phone or secure internet video connection during which medical history and insulin use parameters were obtained and documentation of a physical examination within the prior year was reviewed. Female participants of child-bearing potential were provided a urine pregnancy test. Once enrolled and screened, participants were trained on the use of a Dexcom G6 CGM system (Dexcom, Inc., San Diego, CA) and collected at least 14 days of sensor data and insulin records from home use. Upon completion of the baseline data collection (see below), participants were randomized to the order of the two sessions (with and without BPS). For participants already using a Dexcom G6, retrospective data prior to enrollment could be used. Participants not using a Dexcom G6 CGM were trained and sent equipment for a minimum 14 days of data prior to study admission. Baseline data was used to initialize the controller regarding the participant's TDI as an indicator of insulin needs.

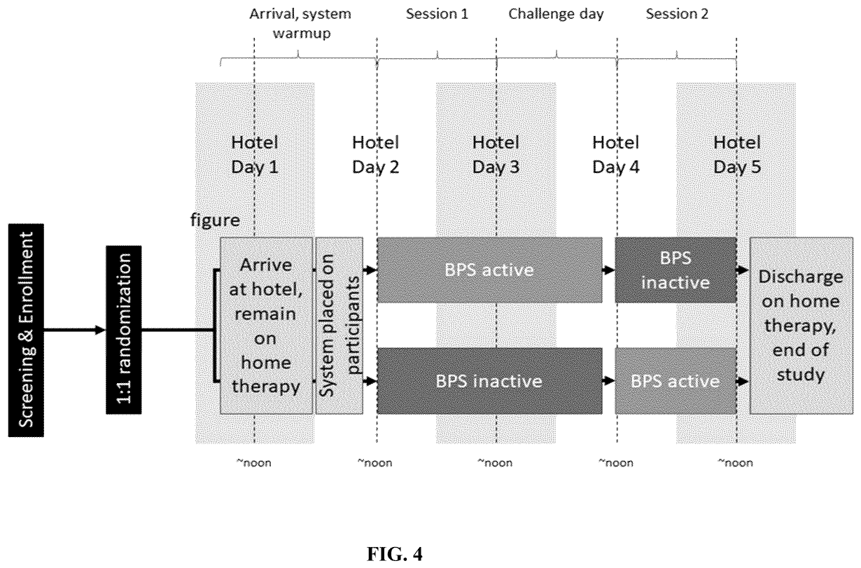

Participants then traveled to the University of Virginia for sequential assessments of the closed loop control modalities in a supervised hotel environment. The timeline for the hotel admission is shown in FIG. 4. The two 24-hour study sessions were designed to be as similar as possible with respect to the timing and content of food and activity: meals had been selected beforehand by participants and repeated for each 24 h period. Breakfast was at 7 am, lunch at noon and dinner at 7 pm. At 10:30 a.m. for each study period, participants together went on a light walk for 25 minutes, approximately 1.5 miles. Participants were otherwise asked not to participate in strenuous exercise during the 24-hour study sessions and if they performed any physical activity to repeat the same routine at the same time during the next study session.

### Challenge Sessions

In between the two 24-hour study sessions was an additional 24-hour period further testing the safety of the BPS system in three different challenge situations: (i) supervised exercise sessions involving HIIT activities, (ii) high carbohydrates high fat meal (pizza), and (iii) low and fast carbohydrate breakfast. Challenges were performed with or without BPS depending on randomization (parallel RCT design).

The challenge exercise sessions were performed to attempt to induce a sharp increase in blood glucose associated with catecholamine response to intense anaerobic activity. Sessions were overseen by a trainer who encouraged participants through bouts of short (up to 1 minute) intense activity separated by 2-minute rest periods. Participants were encouraged to perform up to 6 of these interval bouts. Every 20 seconds during the 1 minute of activity, participants were asked to rate their perceived exertion (RPE) using the 6-20 Borg scale, with the goal of achieving an RPE>17 (“maximal intensity”). Activity types were chosen by participants and included running intervals, climbing stairs, burpees, and exercise bike.

For the pizza challenge, (which occurred 1-2 hours after the HIIT activities) the participants chose the number of pieces of pizza to consume without announcing carbohydrate. Each piece contained 38 g of carbohydrate.

The low and fast-carbohydrate breakfast consisted of drinking juice with 15 g of carbohydrate without announcing the carbohydrate to the system.

### Study Devices

Participants were asked to replace the Dexcom G6 sensor 24-48 hours prior to the first study session. On the morning of the first 24 h study session participants switched therapy to a Tandem t:AP insulin pump (connected t:slim pump, Tandem Diabetes Care, San Diego, CA) using their home insulin parameters. Approximately an hour before the study session, both devices were connected to the DiAs system (University of Virginia, Charlottesville, VA) using a study-provided Android cellphone that allowed for remote monitoring (UVa DWM system). A study blood glucose meter (ContourNext Link; Ascencia Diabetes Care, Parsippany, New Jersey) and study blood ketone meter (Precision Xtra; Abbott, Alameda, CA) were provided to all participants for use as necessary in adherence to the glycemic guidelines. At admission to the hotel, participants were also fitted with a physical activity tracker (Fitbit® Charge 3; Fitbit, San Francisco, CA; data not used by the AID system).

### Study AID System

This study compared two versions of the latest UVA AID system used in FCL: with and without the BPS module enabled. The control algorithms have been described previously. Briefly, this is an MPC system that continually predicts future glycemia and calculates optimal insulin doses to maintain the user at a desired glucose target with a participant's insulin needs informed by current total daily insulin (TDI) use; a minimum of 7 days of CGM and pump information were required for analysis of baseline glycemic control and total daily insulin (TDI) use. This system includes a module running the BPS, an algorithm that determines the probability of a meal-like glucose excursion having occurred in the past 30 minutes and, if above pre-specified thresholds, delivers fixed priming doses based on a user/physician predetermined daily profile and the user total daily insulin needs; in this trial, the profile was fixed at 0% between 11 pm and 6 am, and 6% between 6 am and 11 pm. This module is able to be inactivated to assess AID system function in the absence of the BPS.

### Remote Monitoring and Glycemic Treatment Guidelines

Members of the study team remotely monitored participants' real-time CGM, insulin, and device connectivity data through UVA DiAs web-based monitoring system (DWM) for the entirety of the hotel portion of the study and alerted study nurses, physicians, and technicians regarding glycemic concerns and device connection issues. Treatment decisions were based on CGM data, with SMBG performed only at the recommendation of the medical team. Hypoglycemic treatments were administered with at least one CGM value <60 mg/dL or as requested by the participant following hypoglycemia-related symptoms.

### Outcomes and Statistical Analysis

All glycemic outcomes were computed based on CGM records. CGM data associated with recorded device issues or protocol deviations unrelated to the studied algorithms (e.g. pump occlusion, site failure, CGM disconnection, or prolonged DiAs-pump disconnection) was excluded from the analysis. The primary outcome was percent TIR 70-180 mg/dL during daytime/evening period (6 am-12 am). Per consensus guidelines secondary glycemic outcomes included number of hypoglycemia events (defined as CGM<70 mg/dL for 15 min or longer or the existence of a hypoglycemia treatment); percent time <70 mg/dL; percent time in tight range 80-140; percent time in range 70-180; percent time above range (>180, >250); integrated area under the curve when accounting for starting BG (iAUC); Low Blood Glucose Index (LBGI); High Blood Glucose Index (HBGI); CGM coefficient of variation; and mean CGM glucose value. Secondary outcomes were computed over the 24 h study period, during daytime (6 am-midnight) and nighttime, as well as during the 4 h following a meal.

As the AID system is protected against large insulin injection during or following CGM values lower than 90 mg/dL, we performed a secondary analysis of postprandial periods excluding meals where: (i) minimum glucose value one hour before the meal was lower than 90 mg/dL and (ii) there was a BPS triggered within 4 hours before the meal.

A linear mixed-effects regression model was used for both primary and secondary outcomes (if normally distributed), with the respective pre-randomization variable (e.g. participant characteristic, study phase, etc.) and gender as covariates. Nonparametric Wilcoxon signed-rank tests were used in case of non-normally distributed samples. The significance level was defined as a p-value <0.05 (all p-values are two tailed). Data are reported as mean±standard deviation (SD) if normally distributed and median [IQR]if the distribution is non-normal. Statistical analyses were conducted using SPSS version 28 software. No power analysis was calculated for the design of the clinical trial given the safety and feasibility nature of the pilot study.

### Results

Between 10/20/2022 and 1/15/2023, 15 participants signed informed consent, 2 did not pass screening, and 2 participants withdrew prior to randomization. Thus, 11 were randomized and the trial was completed by 11 (100%) of participants randomized. The demographic characteristics are shown in Table 3. The carbohydrate content for meals on study days, as selected by the participants according to their preference, varied from a mean 74.0 g (absolute range 38-152 g) at dinner, 31.6 g (range 15-65 g) at breakfast, and 43.0 g (range 16-60 g) at lunch. During the pizza challenge, carbohydrate content was 96.7 g (range 38-152 g).

For the comparison between BPS and no BPS, daytime CGM-outcomes were similar with and without BPS: TIR 67.4% [15.1%]vs 65.1% (18.45%) (p=0.43); time-below-range <70 mg/dL (TBR) 0% [0-2.1]vs 0% [0-1.3]; respectively, and this was similarly true for the overall and overnight periods (Table 4). Insulin delivery over the first 3 h after a meal was almost identical 33.5U [26.4-47.0]vs 35.7U [28.7-44.9].

TABLE 3
Baseline characteristics of participants.

| | Demographics | | Value * |
|---|---|---|---|
| Age (years) | | | 38.4 ± 12.9 |
| Weight (kg) | | | 81.2 ± 15.7 |
| Height (cm) | | | 169.7 ± 7 |
| | Biological sex (N, %) | Female | 6, 54.5 |
| | | Male | 5, 45.5 |

| | |
|---|---|
| HbA1c (%) | 6.7 ± 1.1 |
| T1D duration (years) | 21.5 ± 12 |
| Total Daily Insulin Dose (U) | 50.9 ± 23.2 |
| CGM use | 7.6 ± 5.7 |
| AID use | 11.5± 7.2 |

TABLE 4
Glycemic outcomes for Daytime (6 am-12 am), Overnight (12 am-6 am) periods
and Overall (12 pm-12 pm). Data are presented as Median [25^{th}, 75^{th}] percentiles.

| | Daytime | | Overnight | | Overall | |
|---|---|---|---|---|---|---|
| | No BPS | BPS | No BPS | BPS | No BPS | BPS |
| Mean BG | 153 | 152.3 | 117.7 | 113.3 | 146.6 | 145.9 |
| | [144.2, 165.3] | [133.3,171] * | [109.3, 119] | [111.1, 115.2] | [132.2, 151.7] | [128.4 155.9] * |
| % time | 65.7 | 70.6 | 100 | 100 | 74.3 | 75.4 |
| [70-180] | [58.6, 80.6] * | [62.2, 76.5] | [100 100] | [100 100] | [68.9 85.6] * | [70.8 82.4] |
| mg/dL | | | | | | |
| % time | 44.4 | 52.3 | 100 | 93.1 | 58.3 | 61.1 |
| [70-140] | [33, 60.2] * | [41.2, 57.8] * | [83.7 100] | [87.5 100] | [49.1 67.4] | [50.8 68] * |
| mg/dL | | | | | | |
| % time | 0 | 0 | 0 | 0 | 0 | 0 |
| <70 mg/dL | [0, 1.3] | [0, 2.1] | [0 0] | [0 0] | [0 1] | [0 1.6] |
| % time | 0 | 0 | 0 | 0 | 0 | 0 |
| <54 mg/dL | [0, 0] | [0, 0] | [0 0] | [0 0] | [0 0] | [0 0] |
| % time | 34.3 | 25.9 | 0 | 0 | 25.7 | 19.4 |
| >180 | [18.3, 37.8] | [19.8, 37.5] * | [0 0] | [0 0] | [14.4 28.4] | [14.8 28.1] |
| mg/dL | | | | | | |
| % time | 6 | 9.7 | 0 | 0 | 4.2 | 7.3 |
| >250 | [3.2, 15.6] | [2, 15.3] * | [0 0] | [0 0] | [2.3 11.7] * | [1.4 11.8] * |
| mg/dL | | | | | | |
| % CV- | 35.6 | 38.5 | 19.1 | 14.8 | 33.8 | 38.4 |
| glucose | [29.4, 37.5] * | [28.9, 46] * | [12.1 10] * | [9.1 18.9] | [30.5 38.6] * | [29.4 44.7] * |
| % SD- | 52.2 | 59 | 14.5 | 15.1 | 48.9 | 54.2 |
| glucose | [45.8, 60.9] * | [44.5, 70.6] * | [11 23.5] * | [10.1 21.1] * | [44 58.9] * | [43.9 65.5] * |
| (mg/dL) | | | | | | |
| HBGI | 6.6 | 6.5 | 0.4 | 0.3 | 5.1 | 5 |
| | [4, 8] | [3.8, 9.8] * | [0.1 0.7] | [0.1 0.5] | [3.1 6] | [2.9 7.5] * |
| LBGI | 0.4 | 0.6 | 0.2 | 0.2 | 0.4 | 0.5 |
| | [0, 0.7] * | [0.3, 0.8] | [0.1 0.4] | [0.2 0.8] | [0.1 0.7] | [0.4 0.6] |
| Hypo- | 0 | 0 | 0 | 0 | 0 | 1 |
| glycemia | [0, 1] | [0, 1] | [0 0] | [0 0] | [0 1] | [0 1.8] |
| Events | | | | | | |
| Total Daily | 0.4 | 0.4 | 0.07 | 0.08 | 0.47 | 0.45 |
| Insulin | [0.29, 0.45]* | [0.31, 0.47] * | [0.06 0.09] * | [0.05 0.09] * | [0.37 0.52] * | [0.36 0.55] * |
| (U/kg) | | | | | | |
| Total | 0.34 | 0.27 | 0.07 | 0.08 | 0.41 | 0.34 |
| Daily | [0.26, 0.4] * | [0.24, 0.34] * | [0.06 0.09] * | [0.05 0.09] * | [0.35 0.46] * | [0.3 0.42] * |
| Basal | | | | | | |
| Insulin | | | | | | |
| (U/kg) | | | | | | |

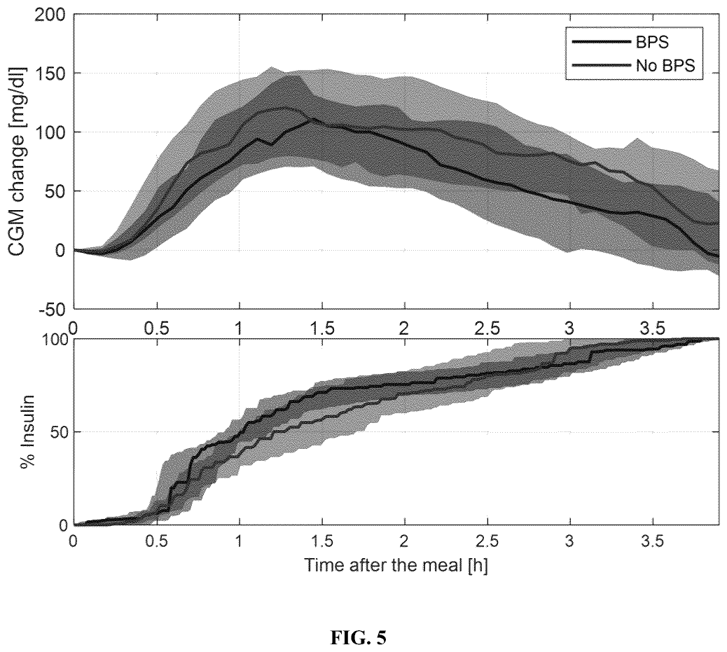

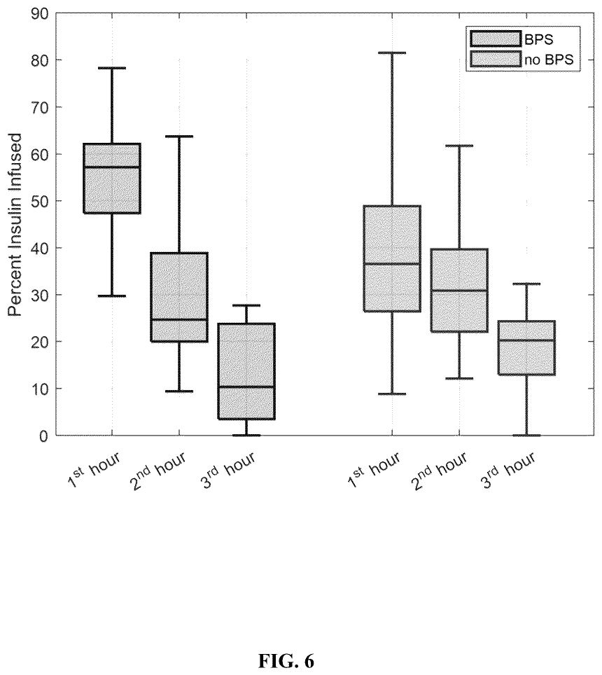

We then selected meals for which CGM values were above 90 mg/dL for an hour prior—to avoid system bias due to hypoglycemia treatments and/or controller BPS attenuation that would remove possibility of BPS firing. This included a total of 43 out of 66 meals (24/19 BPS/no BPS). Postprandial control for BPS vs. no BPS differed with incremental AUC 2530±1934 vs 3228±2029, p<0.047, but CGM outcomes were inconclusive: TIR with 4 hours: 51.2% (33.2%) vs. 40.2% (25.5%) (p=0.24); TBR within 4 hours 0 [0-00]vs 0 [0-0](p=0.2). Difference in glycemia over the postprandial period is further depicted in FIG. 5. These differences in postprandial control can be related to the increased insulin infusion over the first hour after the meal for the system with BPS compared to without it, as shown in FIG. 6. With BPS, 57.24% [50.9%-69.16%] of the insulin is infused over the first hour, compared to 34.71% [11.68%-45.02%] without BPS.

### Challenge Tests of BPS Safety

Tables 5 and 6 provide data on blood glucose around the individual challenges to the BPS systems performed during the day in-between the two 24-hour comparison sessions. During the HIIT sessions and the ensuing 1-2 hours before dinner, TIR was 85.6% (21.2%) for the participants with BPS engaged, and 76.3% (33.4%) for the participants without BPS engaged. Hypoglycemia was experienced by 3/11 participants (all in the group with BPS engaged). Among those with BPS engaged, overall TBR <70 was 0% [0%-11.1%] and TBR <54 was 0% [0%-0%]. There was one participant who in the 90 minutes between exercise and dinner experienced 61.1% TBR <70 and TBR <54 27.78% by CGM, though at the time of CGM nadir of 45 mg/dL, fingerstick blood glucose was 64 mg/dL, and fingerstick blood glucose had returned above 70 mg/dL at least 10 minutes earlier than CGM. This participant belonged to the BPS group, but no BPS bolus was triggered during the HIIT session. In the group with BPS engaged, a BPS bolus was triggered for 3/7 participants, with 2 of these participants experiencing hypoglycemia within 2 hours; for these 2 participants during the time before dinner, TBR <70 was 13.3 and 8.8% and TBR <54 was 0% and 0%.

TABLE 5
Challenge test Results

| | | | | | TBR | | | | | | tbr |
|---|---|---|---|---|---|---|---|---|---|---|---|
| | | | | | <70 | | | | TIR | | <70 after |
| | PID | | | | during/ | BPS | Grams | | <70 after | TIR | orange |
| | (to be | BPS | Activity | TIR | after | activ- | CHO | TIR | pizza | after | juice |
| | removed | active | during | during/ | HIIT | ation | in | after | challenge | orange | challenge |
| Participant | before | or | HIIT | after | (BG | during | pizza | pizza | (# | juice | (# |
| number | publication | silent? | challenge | HIIT | nadir) | HIIT | challenge | challenge | episodes) | challenge | episodes) |
| 1 | 91101 | 0 | Bike | 100.0 | 0 (106) | — | 152 | 53.1 | 0 (0) | 100.0 | 0 (0) |
| 2 | 91102 | 1 | Bike | 100.0 | 0 (108) | 0 | 152 | 42.7 | 0 (0) | 100.0 | 0 (0) |
| 3 | 91103 | 1 | Shuttle | 38.9 | 61.1 (45) | 0 | 76 | 54.2 | 12.5 (3) | 60.4 | 0 (0) |
| | | | run | | | | | | | | |
| 4 | 91105 | 1 | Shuttle | 92.9 | 0 (82) | 1 | 76 | 59.4 | 8.3 (2) | 89.6 | 0 (0) |
| | | | run | | | | | | | | |
| 5 | 91106 | 1 | Shuttle | 86.7 | 13.3 (62) | 1 | 152 | 57.3 | 0 (0) | 89.6 | 0 (0) |
| | | | run | | | | | | | | |
| 6 | 91108 | 0 | Walking | 29.2 | 0 (164) | — | 38 | 50.0 | 0 (0) | 12.5 | 0 (0) |
| | | | inclined | | | | | | | | |
| | | | treadmill | | | | | | | | |
| 7 | 91111 | 0 | Stair run | 100.0 | 0 (71) | — | 76 | 66.7 | 0 (0) | 68.8 | 0 (0) |
| 8 | 91112 | 1 | Shuttle | 89.5 | 0 (124) | 0 | 76 | 59.4 | 3.1 (2) | 100.0 | 0 (0) |
| | | | run | | | | | | | | |
| 9 | 91113 | 1 | Bicycle | 100.0 | 0 (92) | 0 | 114 | 53.1 | 0 (0) | 83.3 | 0 (0) |
| 10 | 91114 | 0 | Burpees | 76.2 | 0 (78) | | 76 | 79.2 | 0 (0) | 58.3 | 0 (0) |
| 11 | 91115 | 1 | Stair run | 91.2 | 8.8 (65) | 1 | 76 | 56.3 | 0 (0) | 100.0 | 0 (0) |

TABLE 6
Challenge test Results

| | | | | | Number of |
|---|---|---|---|---|---|
| | | | | Number of | Hypoglycemia |
| | | | | Hypoglycemia | Events (BG nadir |
| | | BPS | BPS | Events (BG nadir | in mg/dL) |
| Participant | | Bolus, | Bolus, | in mg/dL) within | within 4 h - BPS |
| number | Meal | units | % TDI^{†} | 4 h - BPS engaged | not engaged |
| 1 | Lunch | 4.21 | 6.10 | 0 (151) | 0 (184) |
| | Dinner | 4.15 | 6.01 | 0 (107) | 0 (162) |
| | Breakfast | 2.90 | 4.20 | 0 (126) | 0 (120) |
| 2 | Lunch | 5.94 | 6.00 | 0 (153) | 0 (115) |
| | Dinner | 4.17 | 4.21 | 0 (146) | 0 (141) |
| | Breakfast | 5.97 | 6.03 | 0 (155) | 0 (119) |
| 3 | Lunch | 2.77 | 6.02 | 0 (100) | 0 (99) |
| | Dinner | 1.94 | 4.21 | 0 (117) | 0 (91) |
| | Breakfast | 2.78 | 6.04 | 3 (50) | 0 (86) |
| 4 | Lunch | 1.20 | 5.73 | 0 (95) | — (—) |
| | Dinner** | 0.00 | 0.00 | 0 (94) | 0 (77) |
| | Breakfast | 1.28 | 6.10 | 0 (101) | 0 (72) |
| 5 | Lunch*** | 0.00 | 0.00 | 2 (39) | 0 (103) |
| | Dinner | 1.81 | 4.20 | 0 (107) | 0 (103) |
| | Breakfast | 2.58 | 6.00 | 1 (67) | 0 (140) |
| 6 | Lunch | 0.88 | 4.20 | 0 (195) | 0 (196) |
| | Dinner | 0.88 | 4.20 | 0 (217) | 0 (235) |
| | Breakfast | 1.26 | 6.00 | 0 (171) | 0 (221) |
| 7 | Lunch | 2.88 | 6.00 | 0 (154) | 0 (160) |
| | Dinner | 2.02 | 4.20 | 0 (143) | 0 (116) |
| | Breakfast | 2.02 | 4.20 | 0 (111) | 0 (122) |
| 8 | Lunch | 0.00 | 0.00 | 0 (209) | 0 (174) |
| | Dinner | 0.00 | 0.00 | 0 (79) | 0 (163) |
| | Breakfast | 5.10 | 6.00 | 0 (108) | 0 (95) |
| 9 | Lunch | — | — | — (—) | — (—) |
| | Dinner* | 1.18 | 3.11 | 0 (114) | 0 (130) |
| | Breakfast | 2.28 | 6.00 | 0 (77) | 1 (64) |
| 10 | Lunch | 2.89 | 6.03 | 0 (83) | 0 (85) |
| | Dinner | 2.89 | 6.02 | 0 (128) | 0 (130) |
| | Breakfast | 3.03 | 6.31 | 0 (87) | 0 (96) |
| 11 | Lunch | 2.52 | 6.00 | 0 (156) | 0 (245) |
| | Dinner** | 0.00 | 0.00 | 0 (84) | 0 (174) |
| | Breakfast | 2.52 | 6.01 | 0 (140) | 0 (171) |

During the pizza challenge and 8 hours afterward, TIR among participants with BPS engaged was 54.6% (5.8%), TAR was 42.7% [35.4%-45.3%], TBR <70 was 0% [0%-5.73%] and TBR <54 was 0% [0%-0%]. There was a BPS bolus for all 7 participants with BPS engaged, with one resulting in hypoglycemia (nadir BG was 49 mg/dL).

During the orange juice challenge and 4 hours afterward, TIR when BPS was engaged was 89% (14.2%). TAR was 11% (14.2%), TBR <70 was 0% [0%-0%]. Among participants without BPS engaged, TIR was 63.5% [46.9-62.1%]. TAR was 36.5% [23.4%-53.1%], TBR <70 was 0% [0%-0%]. Out of the 7 participants that had BPS engaged through this challenge, there was a BPS bolus for 5 of them, none of them experiencing hypoglycemia during the 4 hours after the bolus.

### DISCUSSION

Mealtime blood glucose excursions have been particularly problematic to AID systems used as FCL due to mismatches between rapid carbohydrate absorption and slower insulin injection and absorption. We found that while a system designed to deliver a rapid insulin bolus following meal-like glucose disturbance did not increase overall TIR (largely from high variability between participants), this system resulted in reduced glycemic iAUC during episodes where a BPS dose was possible from current glycemic circumstances. This provides proof of concept that a rapid insulin dose early after carbohydrate ingestion could mitigate some of the rise in glucose before standard controller-based insulin delivery is given. In this current test of two 24-hour periods, only 65% of meals were eligible to receive a BPS based on pre-meal glycemia and IOB. If these results were maintained over a longer timeframe, it would be expected that BPS would result in increased TIR.

Because of legitimate concerns about the safety of such a system in real-world circumstances, we additionally tested three situations that could increase risk of hypoglycemia should the system provide an excess amount of insulin outside of when a meal was ingested. This included times when only a small amount of simple sugar was ingested (e.g. orange juice for breakfast), when the glucose level increased due to intense physical activity (HIIT), and after a high-fat, high-carbohydrate meal (e.g. pizza). Our concern in testing these was that the insulin delivered by BPS would be in significant excess of physiologic demand and produce dangerous hypoglycemia. While we noted hypoglycemia in the time following these episodes, only one participant had TBR <54 and no one required two treatments, including during episodes where a BPS bolus was delivered during intense exercise. This was reassuring that the system's response to dropping glucose levels after a BPS dose (e.g., reducing insulin delivery following the drop in glucose level) was adequate to avoid severe hypoglycemia. Still, further testing in real-world settings is needed since many additional problematic challenges to this system exist.

This system resulted in overall TIR that was above the recommended ADA threshold of 70% despite being used in FCL without carbohydrate announcement. This compares to TIR of other insulin-only AID systems used in FCL of 58-78%. While current goals are to allow for adequate management without as much user interaction, we previously found that this AID system did maintain better glycemic control with carbohydrate announcement, with an additional 9% TIR. Still, this type of system could be particularly beneficial for users who omit meal announcement, which occurs regularly in some settings. Currently-available AID systems are not able to adjust rapidly enough to post-prandial glycemic excursions, underscoring the need to develop new systems such as this.

This study benefited from a randomized cross-over design in which each participant served as his/her own control. Limitations include that we tested three separate challenges to the system, while clearly additional challenges of stressors and timing of food and exercise could pose further problems. Also, the highly-supervised nature of this study was unlike real-world situations, where episodes of BPS firing may occur without a user noticing, increasing risks of delayed response and increased hypoglycemia.

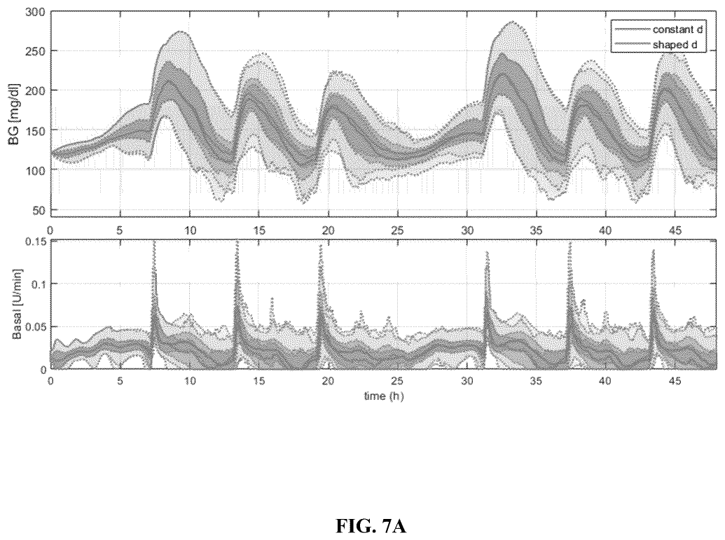

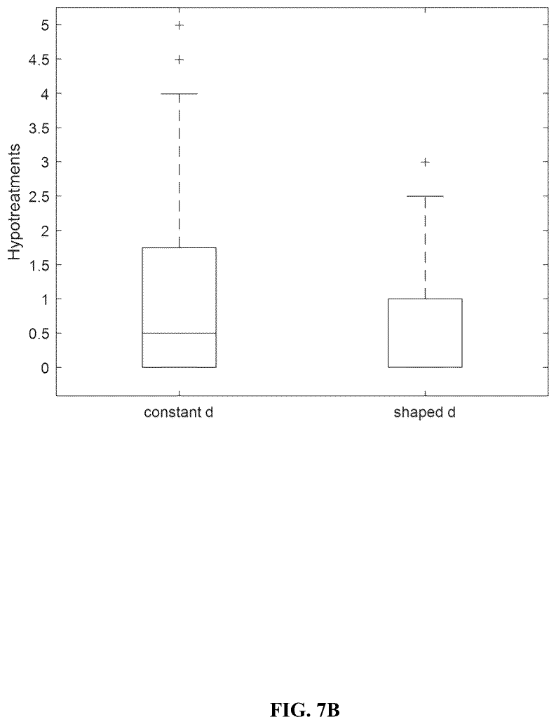

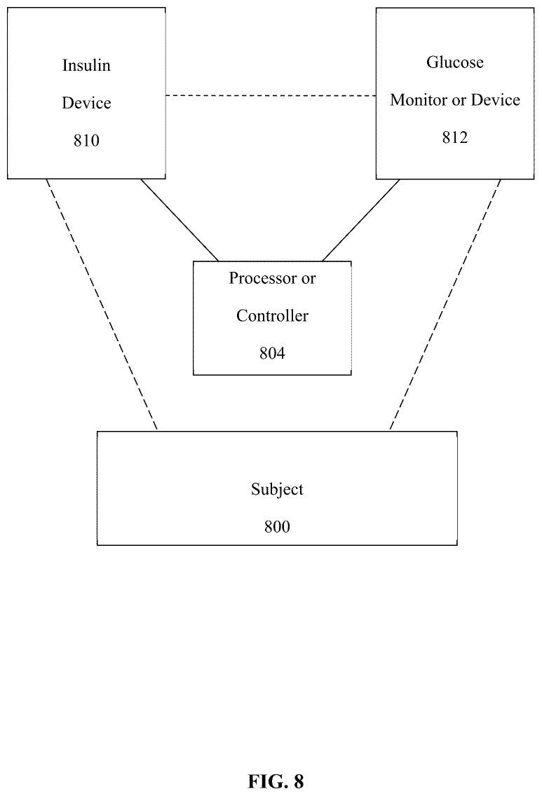

Referring to FIGS. 7-8, in conclusion, we found that a BPS system resulted in a reduction in iAUC glycemic levels when comparing meals where there was potential for BPS to act. These data raise the possibility that a system like this could be incorporated into an AID controller to reduce hyperglycemia after unannounced meals—possibly moving away from completely hybrid closed loop systems and allowing an additional degree of freedom for people with TlD.

FIGS. 8-12 are exemplary system architectures, functional block diagrams, etc. that can be used for embodiments of the system 100, processor 102, or devices 108 disclosed herein.

As shown in FIG. 8, a processor 804 or controller communicates with the glucose monitor or data source 812, and optionally an insulin delivery device (e.g., other device 810). The glucose monitor or device communicates with the subject 800 to monitor glucose levels of the subject 800. The processor 804 or controller is configured to perform the required calculations. Optionally, the insulin delivery device communicates with the subject 800 to deliver insulin to the subject 800. The processor 804 or controller is configured to perform the required calculations. The glucose monitor and the insulin delivery device may be implemented as a separate device or as a single device. The processor 804 can be implemented locally in the glucose monitor, the insulin delivery device, or a standalone device (or in any combination of two or more of the glucose monitor, insulin device, or a stand along device). The processor 804 or a portion of the system can be located remotely such that the device is operated as a telemedicine device.

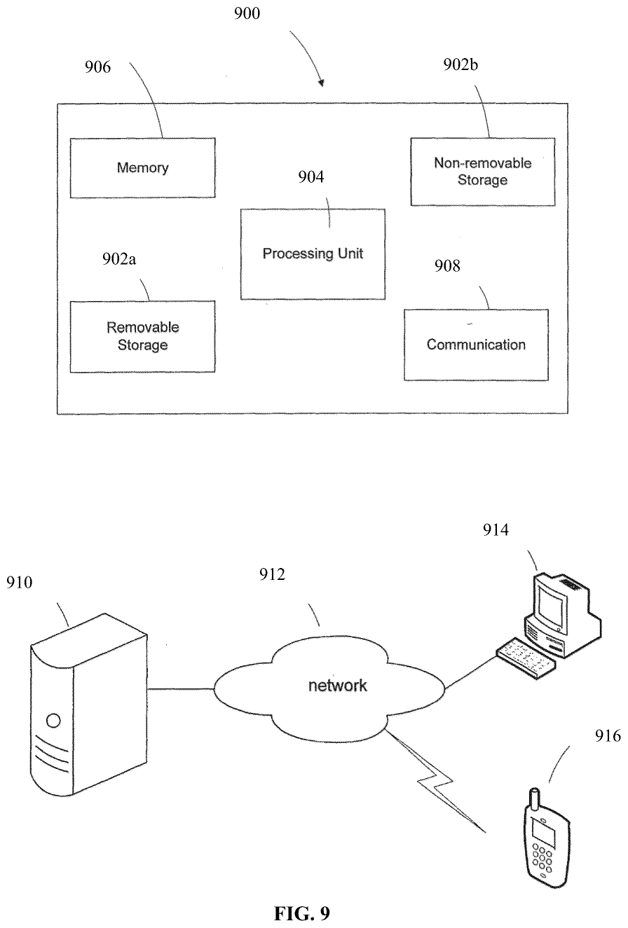

Referring to FIG. 9, in its most basic configuration, computing device 900 typically includes at least one processor 904 and memory 906. Depending on the exact configuration and type of computing device, memory 906 can be volatile (such as RAM), non-volatile (such as ROM, flash memory, etc.) or some combination of the two.

Additionally, the computing device 900 may also have other features and/or functionality. For example, the computing device 900 could also include additional removable and/or non-removable storage including, but not limited to, magnetic or optical disks or tape, as well as writable electrical storage media. Such additional storage is shown the figure by removable storage 902a and non-removable storage 902b. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The computing device 900 may also contain one or more communications connections 908 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In addition to a stand-alone computing machine, embodiments of the invention can also be implemented on a network system comprising a plurality of computing devices that are in communication with a networking means, such as a network with an infrastructure or an ad hoc network. The network connection can be wired connections or wireless connections. As a way of example, FIG. 9 illustrates a network system in which embodiments of the invention can be implemented. In this example, the network system comprises computer 910 (e.g. a network server), network connection means 912 (e.g. wired and/or wireless connections), computer terminal 914, and PDA (e.g. a smart-phone) 916 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module 914 may be glucose monitor device. In an embodiment, it should be appreciated that the module listed as 914 may be a glucose monitor device, artificial pancreas, and/or an insulin device (or other interventional or diagnostic device). Any of the components may be multiple in number. The embodiments of the invention can be implemented in anyone of the devices of the system. For example, execution of the instructions or other desired processing can be performed on the same computing device 900. Alternatively, an embodiment of the invention can be performed on different computing devices of the network system. For example, certain desired or required processing or execution can be performed on one of the computing devices of the network (e.g., server 910 and/or glucose monitor device), whereas other processing and execution of the instruction can be performed at another computing device (e.g., terminal 914) of the network system, or vice versa. In fact, certain processing or execution can be performed at one computing device (e.g. server 910 and/or insulin device, artificial pancreas, or glucose monitor device (or other interventional or diagnostic device)); and the other processing or execution of the instructions can be performed at different computing devices that may or may not be networked. For example, the certain processing can be performed at terminal 914, while the other processing or instructions are passed to a computing device 900 where the instructions are executed. This scenario may be of particular value especially when the PDA device, for example, accesses to the network through computer terminal 914 (or an access point in an ad hoc network). For another example, software to be protected can be executed, encoded or processed with one or more embodiments of the invention. The processed, encoded or executed software can then be distributed to customers. The distribution can be in a form of storage media (e.g., disk) or electronic copy.

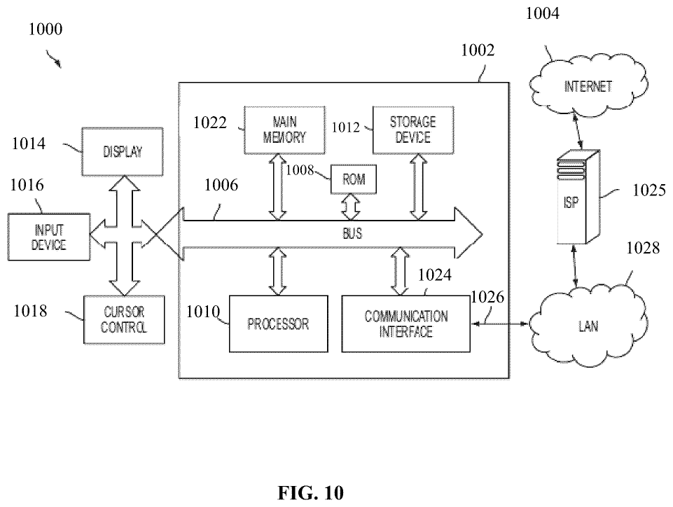

FIG. 10 is a block diagram that illustrates a system 1000 including a computer system 1002 and the associated Internet 1004 connection upon which an embodiment may be implemented. Such configuration is typically used for computers (hosts) connected to the Internet 1004 and executing a server or a client (or a combination) software. A source computer such as laptop, an ultimate destination computer and relay servers, for example, as well as any computer or processor described herein, may use the computer system configuration and the Internet connection shown in FIG. 10. The system 1004 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 10 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. The computer system of FIG. 10 may, for example, be an Apple Macintosh computer or Power Book, or an IBM compatible PC. Computer system 1000 includes a bus 1006, an interconnect, or other communication mechanism for communicating information, and a processor, commonly in the form of an integrated circuit, coupled with bus 1006 for processing information and for executing the computer executable instructions. Computer system 1000 also includes a main memory 1008, such as a Random Access Memory (RAM) or other dynamic storage device, coupled to bus 1006 for storing information and instructions to be executed by processor 1010.

Main memory 1008 also may be used for storing temporary variables or other intermediate information during execution of instructions to be executed by processor 1010. Computer system 1000 further includes a Read Only Memory (ROM) 1008 (or other non-volatile memory) or other static storage device coupled to bus 1006 for storing static information and instructions for processor 1010. A storage device 1012, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 1006 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 1000 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 1000 may be coupled via bus 1006 to a display 1014, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 1016, including alphanumeric and other keys, is coupled to bus 1006 for communicating information and command selections to processor 1010. Another type of user input device is cursor control 1018, such as a mouse, a trackball, or cursor direction keys for communicating direction information and command selections to processor 1010 and for controlling cursor movement on display 1014. This input device typically has two degrees of freedom in two axes, a first axis (e.g., x) and a second axis (e.g., y), that allows the device to specify positions in a plane.

The computer system 1002 may be used for implementing the methods and techniques described herein. According to one embodiment, those methods and techniques are performed by computer system 1002 in response to processor 1010 executing one or more sequences of one or more instructions contained in main memory 1020. Such instructions may be read into main memory 1022 from another computer-readable medium, such as storage device 1012. Execution of the sequences of instructions contained in main memory 1022 causes processor 1010 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 1010) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 1006. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 1010 for execution. For example, the instructions may initially be carried on a magnetic disk of a remote computer. The remote computer can load the instructions into its dynamic memory and send the instructions over a telephone line using a modem. A modem local to computer system 1000 can receive the data on the telephone line and use an infra-red transmitter to convert the data to an infra-red signal. An infra-red detector can receive the data carried in the infra-red signal and appropriate circuitry can place the data on bus 1006. Bus 1006 carries the data to main memory 1022, from which processor 1010 retrieves and executes the instructions. The instructions received by main memory 1022 may optionally be stored on storage device 1012 either before or after execution by processor 1010.

Computer system 1000 also includes a communication interface 1024 coupled to bus 1006. Communication interface 1024 provides a two-way data communication coupling to a network link 1026 that is connected to a local network 1028. For example, communication interface 1024 may be an Integrated Services Digital Network (ISDN) card or a modem to provide a data communication connection to a corresponding type of telephone line. As another non-limiting example, communication interface 1024 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 1818 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. In any such implementation, communication interface 1024 sends and receives electrical, electromagnetic or optical signals that carry digital data streams representing various types of information.

Network link 1026 typically provides data communication through one or more networks to other data devices. For example, network link 1026 may provide a connection through local network 1028 to a host computer or to data equipment operated by an Internet Service Provider (ISP) 1025. ISP 1025 in turn provides data communication services through the world wide packet data communication network Internet 1004. Local network 1028 and Internet 1004 both use electrical, electromagnetic or optical signals that carry digital data streams. The signals through the various networks and the signals on the network link 1026 and through the communication interface 1024, which carry the digital data to and from computer system 1000, are exemplary forms of carrier waves transporting the information.

A received code may be executed by processor 1010 as it is received, and/or stored in storage device 1012, or other non-volatile storage for later execution. In this manner, computer system 1000 may obtain application code in the form of a carrier wave.

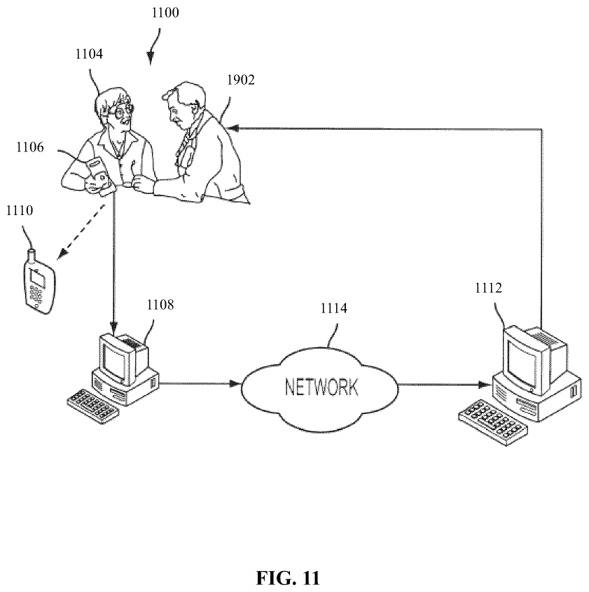

FIG. 11 illustrates a system in which one or more embodiments of the invention can be implemented using a network, or portions of a network or computers. Although the present invention glucose monitor, artificial pancreas or insulin device (or other interventional or diagnostic device) may be practiced without a network. FIG. 11 diagrammatically illustrates an exemplary system in which examples of the invention can be implemented. In an embodiment the glucose monitor, artificial pancreas or insulin device (or other interventional or diagnostic device) may be implemented by the subject (or patient) locally at home or other desired location. However, in an alternative embodiment it may be implemented in a clinic setting or assistance setting. For instance, a clinic setup 1100 provides a place for doctors (e.g. 1102) or clinician/assistant to diagnose patients (e.g. 1104) with diseases related with glucose and related diseases and conditions. A glucose monitoring device 1106 can be used to monitor and/or test the glucose levels of the patient—as a standalone device. It should be appreciated that while only glucose monitor device 1106 is shown in the figure, the system of the invention and any component thereof may be used in the manner depicted by FIG. 11. The system or component may be affixed to the patient or in communication with the patient as desired or required. For example the system or combination of components thereof—including a glucose monitor device 1106 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g. clinical visit) or long term (e.g. clinical stay or family). The glucose monitoring device outputs can be used by the doctor (clinician or assistant) for appropriate actions, such as insulin injection or food feeding for the patient, or other appropriate actions or modeling. Alternatively, the glucose monitoring device output can be delivered to computer terminal 1112 for instant or future analyses. The delivery can be through cable or wireless or any other suitable medium. The glucose monitoring device output from the patient can also be delivered to a portable device, such as PDA 1110. The glucose monitoring device outputs with improved accuracy can be delivered to a glucose monitoring center 1112 for processing and/or analyzing. Such delivery can be accomplished in many ways, such as network connection 1114, which can be wired or wireless.

In addition to the glucose monitoring device outputs, errors, parameters for accuracy improvements, and any accuracy related information can be delivered, such as to computer and/or glucose monitoring center 1112 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

Examples of the invention can also be implemented in a standalone computing device associated with the target glucose monitoring device, artificial pancreas, and/or insulin device (or other interventional or diagnostic device.

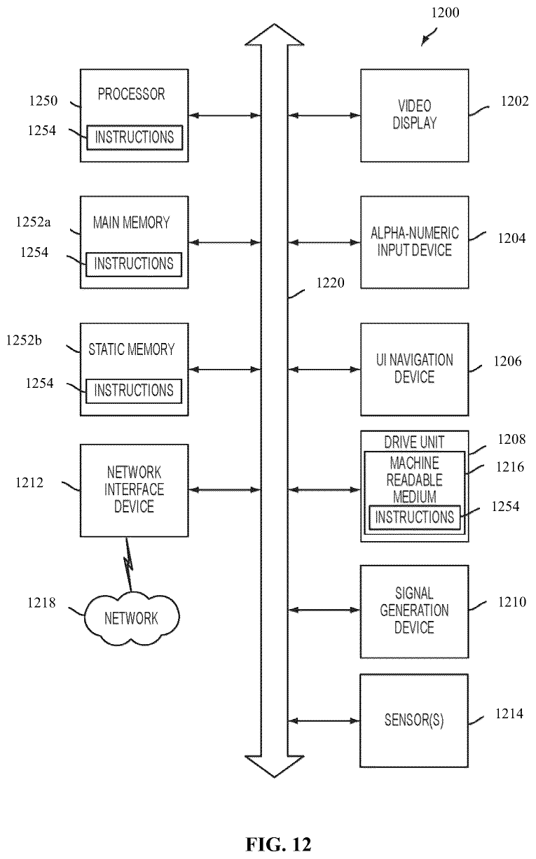

Referring to FIG. 12, an aspect of an embodiment of the present invention includes, but not limited thereto, a system, method, and computer readable medium, which illustrates a block diagram of an example machine 1200 upon which one or more embodiments (e.g., discussed methodologies) can be implemented (e.g., run).

FIG. 12 illustrates a block diagram of an example machine 1200 upon which one or more embodiments (e.g., discussed methodologies) can be implemented (e.g., run).

Examples of machine 1200 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1200) and software architectures that can be deployed in example embodiments.

In an example, the machine 1200 can operate as a standalone device or the machine 1200 can be connected (e.g., networked) to other machines.

In a networked deployment, the machine 1200 can operate in the capacity of either a server or a client machine in server-client network environments. In an example, machine 1200 can act as a peer machine in peer-to-peer (or other distributed) network environments. The machine 1200 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1200. Further, while only a single machine 1200 is illustrated, the term “machine” shall also be taken to include any collection of machines that individually or jointly execute a set (or multiple sets) of instructions to perform any one or more of the methodologies discussed herein.

Example machine (e.g., computer system) 1200 can include a processor 1250 (e.g., a central processing unit (CPU), a graphics processing unit (GPU) or both), a main memory 1252a and a static memory 1252b, some or all of which can communicate with each other via a bus 1220. The machine 1200 can further include a display unit 1202, an alphanumeric input device 1204 (e.g., a keyboard), and a user interface (UI) navigation device 1206 (e.g., a mouse). In an example, the display unit 1202, input device 1204 and UI navigation device 1206 can be a touch screen display. The machine 1200 can additionally include a storage device (e.g., drive unit) 1208, a signal generation device 1210 (e.g., a speaker), a network interface device 1212, and one or more sensors 1214, such as a global positioning system (GPS) sensor, compass, accelerometer, or other sensor.

The storage device 1208 can include a machine readable medium 1216 on which is stored one or more sets of data structures or instructions 1254 (e.g., software) embodying or utilized by any one or more of the methodologies or functions described herein. The instructions 1254 can also reside, completely or at least partially, within the main memory 1252a, within static memory 1252b, or within the processor 804 during execution thereof by the machine 1200. In an example, one or any combination of the processor 804, the main memory 1252a, the static memory 1252b, or the storage device 1208 can constitute machine readable media.

While the machine readable medium 1216 is illustrated as a single medium, the term “machine readable medium” can include a single medium or multiple media (e.g., a centralized or distributed database, and/or associated caches and servers) that configured to store the one or more instructions 1254. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1254 can further be transmitted or received over a communications network 1218 using a transmission medium via the network interface device utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### REFERENCES

The following patents, applications and publications as listed below and throughout this document are hereby incorporated by reference in their entirety herein, and which are not admitted to be prior art with respect to the present invention by inclusion in this section.
1. Brown S A, Kovatchev B P, Raghinaru D, et al. Six-Month Randomized, Multicenter Trial of Closed-Loop Control in Type 1 Diabetes. N Engl J Med 2019; 381(18): 1707-17.
2. Breton M D, Kanapka L G, Beck R W, et al. A Randomized Trial of Closed-Loop Control in Children with Type 1 Diabetes. N Engl J Med 2020; 383(9): 836-45.
3. Wadwa R P, Reed Z W, Buckingham B A, et al. Trial of Hybrid Closed-Loop Control in Young Children with Type 1 Diabetes. N Engl J Med 2023; 388(11): 991-1001.
4. Russell S J, Beck R W, Damiano E R, et al. Multicenter, Randomized Trial of a Bionic Pancreas in Type 1 Diabetes. N Engl J Med 2022; 387(13): 1161-72.
5. Bally L, Thabit H, Kojzar H, et al. Day-and-night glycaemic control with closed-loop insulin delivery versus conventional insulin pump therapy in free-living adults with well controlled type 1 diabetes: an open-label, randomised, crossover study. Lancet Diabetes Endocrinol 2017; 5(4): 261-70.
6. Ware J, Allen J M, Boughton C K, et al. Randomized Trial of Closed-Loop Control in Very Young Children with Type 1 Diabetes. N Engl J Med 2022; 386(3): 209-19.
7. Ware J, Boughton C K, Allen J M, et al. Cambridge hybrid closed-loop algorithm in children and adolescents with type 1 diabetes: a multicentre 6-month randomised controlled trial. Lancet Digit Health 2022; 4(4): e245-e55.
8. Templer S. Closed-Loop Insulin Delivery Systems: Past, Present, and Future Directions. Front Endocrinol (Lausanne) 2022; 13:919942.
9. Cameron F M, Ly T T, Buckingham B A, et al. Closed-Loop Control Without Meal Announcement in Type 1 Diabetes. Diabetes Technol Ther 2017; 19(9): 527-32.
10. Garcia-Tirado J, Diaz J L, Esquivel-Zuniga R, et al. Advanced Closed-Loop Control System Improves Postprandial Glycemic Control Compared With a Hybrid Closed-Loop System Following Unannounced Meal. Diabetes Care 2021.
11. Dovc K, Piona C, Yeşiltepe Mutlu G, et al. Faster Compared With Standard Insulin Aspart During Day-and-Night Fully Closed-Loop Insulin Therapy in Type 1 Diabetes: A Double-Blind Randomized Crossover Trial. Diabetes Care 2020; 43(1): 29-36.
12. Tsoukas M A, Majdpour D, Yale J F, et al. A fully artificial pancreas versus a hybrid artificial pancreas for type 1 diabetes: a single-centre, open-label, randomised controlled, crossover, non-inferiority trial. Lancet Digit Health 2021; 3(11): e723-e32.
13. Cherñavvsky D R, DeBoer M D, Keith-Hynes P, et al. Use of an artificial pancreas among adolescents for a missed snack bolus and an underestimated meal bolus. Pediatr Diabetes 2016; 17(1): 28-35.
14. Paldus B, Morrison D, Zaharieva D P, et al. A Randomized Crossover Trial Comparing Glucose Control During Moderate-Intensity, High-Intensity, and Resistance Exercise With Hybrid Closed-Loop Insulin Delivery While Profiling Potential Additional Signals in Adults With Type 1 Diabetes. Diabetes Care 2022; 45(1): 194-203.
15. Borg G A. Psychophysical bases of perceived exertion. Med Sci Sports Exerc 1982; 14(5): 377-81.
16. Garcia-Tirado J, Lv D, Corbett J P, Colmegna P, Breton M D. Advanced hybrid artificial pancreas system improves on unannounced meal response—In silico comparison to currently available system. Comput Methods Programs Biomed 2021; 211:106401.
17. Corbett J P, Garcia-Tirado J, Colmegna P, Diaz Castaneda J L, Breton M D. Using an Online Disturbance Rejection and Anticipation System to Reduce Hyperglycemia in a Fully Closed-Loop Artificial Pancreas System. J Diabetes Sci Technol 2022; 16(1): 52-60.
18. Battelino T, Danne T, Bergenstal R M, et al. Clinical Targets for Continuous Glucose Monitoring Data Interpretation: Recommendations From the International Consensus on Time in Range. Diabetes Care 2019; 42(8): 1593-603.
19. Garcia-Tirado J, Colmegna P, Villard O, et al. Assessment of Meal Anticipation for Improving Fully Automated Insulin Delivery in Adults With Type 1 Diabetes. Diabetes Care 2023; 46(9): 1652-8.
20. Jaser S S, Datye K A. Frequency of Missed Insulin Boluses in Type 1 Diabetes and Its Impact on Diabetes Control. Diabetes Technol Ther 2016; 18(6): 341-2.

It will be understood that modifications to the embodiments disclosed herein can be made to meet a particular set of design criteria. For instance, any of the components, features, or steps of the system, apparatus, or method can be any suitable number or type of each to meet a particular objective. Therefore, while certain exemplary embodiments of the systems and methods disclosed herein have been discussed and illustrated, it is to be distinctly understood that the invention is not limited thereto but can be otherwise variously embodied and practiced within the scope of the following claims.

It will be appreciated that some components, features, and/or configurations can be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiments. Thus, the components, features, and/or configurations of the various embodiments can be combined in any manner and such combinations are expressly contemplated and disclosed by this statement

It will be appreciated by those skilled in the art that the present invention can be embodied in other specific forms without departing from the spirit or essential characteristics thereof. The presently disclosed embodiments are therefore considered in all respects to be illustrative and not restrictive. The scope of the invention is indicated by the appended claims rather than the foregoing description and all changes that come within the meaning, range, and equivalence thereof are intended to be embraced therein. Additionally, the disclosure of a range of values is a disclosure of every numerical value within that range, including the end points.

What is claimed is:

1. An insulin delivery controller which implements a processor configuration to efficiently attain an insulin delivery target, the insulin delivery controller comprising:
a processor and a memory associated with the processor, the memory including instructions stored thereon that when executed by the processor will cause the processor to:
process glucose data received from the memory, including a data representation of glycemic disturbance (d(t));
determine a glucose rate of change (G′(t));
generate a command signal to dynamically reshape a glycemic disturbance within a prediction horizon of the insulin delivery controller according to the G′(t); and
generate an insulin command signal for an insulin delivery unit to adjust an insulin delivery dosage amount and/or an insulin delivery dosage rate.

2. The insulin delivery controller of claim 1 in combination with an insulin delivery unit, wherein:
the insulin delivery unit includes a processor and a memory with instructions stored thereon that when executed by the insulin delivery processor will cause the insulin delivery processor to generate an insulin delivery dose based on the insulin delivery dosage amount and/or the insulin delivery dosage rate.

3. The insulin delivery controller of claim 1, wherein:
instructions will cause the processor to hold d(t) constant or decrease d(t) based on a threshold (G′_{Lim}).

4. The insulin delivery controller of claim 3, wherein:
instructions will cause the processor to modulate the decrease of d(t) based on a threshold (G̅′̅).

5. The insulin delivery controller of claim 1, wherein:
instructions will cause the processor to estimate d(t).

6. The insulin delivery controller of claim 5, wherein:
instructions will cause the processor to estimate d(t) periodically.

7. The insulin delivery controller of claim 5, wherein:
instructions will cause the processor to estimate d(t) every five minutes.

8. The insulin delivery controller of claim 5, wherein:
instructions will cause the processor to estimate d(t) using a Kalman filtering technique.

9. The insulin delivery controller of claim 1, wherein:
instructions will cause the processor to process the glucose data, determine the (G′(t)), and generate the command signal to dynamically reshape the glycemic disturbance via a closed loop control (CLC) process.

10. The insulin delivery controller of claim 9, wherein:
instructions will cause the processor to implement, as part of the CLC process:
a model predictive control (MPC) algorithm;
a proportional integral derivative (PID) algorithm; or
a fuzzy logic (FL) algorithm.

11. The insulin delivery controller of claim 10, wherein:
instructions will cause the processor to generate the insulin command signal for an insulin delivery unit that adjusts the insulin delivery dosage amount and/or the insulin delivery dosage rate to modulate insulin delivery, wherein insulin delivery will be modulated to:
minimize a risk of or prevent hypoglycemia; and/or
attenuate glycemic disturbances tending towards hyperglycemia.

12. The insulin delivery controller of claim 10, wherein:
instructions will cause the processor to generate the insulin command signal for an insulin delivery unit that adjusts the insulin delivery dosage amount and/or the insulin delivery dosage rate to modulate insulin delivery, wherein insulin delivery will be modulated to:
approach a target glucose level.

13. A computer readable medium including instructions stored thereon that when executed by a processor will cause the processor to efficiently attain an insulin delivery target by:
processing glucose data including a data representation of glycemic disturbance (d(t));
determining a glucose rate of change (G′(t));
generating a command signal to dynamically reshape a glycemic disturbance within a prediction horizon of an insulin delivery controller according to the G′(t); and
generating an insulin command signal for an insulin delivery unit to adjust an insulin delivery dosage amount and/or an insulin delivery dosage rate.

14. The computer readable medium of claim 13, wherein:
processing the glucose data, determining the (G′(t)), and generating the command signal to dynamically reshape the glycemic disturbance are performed via a closed loop control (CLC) process.

15. The computer readable medium of claim 14, wherein:
the CLC process includes:
a model predictive control (MPC) algorithm;
a proportional integral derivative (PID) algorithm; or
a fuzzy logic (FL) algorithm.

16. The computer readable medium of claim 15, wherein:
generating the insulin command signal for an insulin delivery unit includes generating the insulin command signal so that it will adjust the insulin delivery dosage amount and/or the insulin delivery dosage rate to modulate insulin delivery, wherein insulin delivery is modulated to:
minimize a risk of or prevent hypoglycemia; and/or
attenuate glycemic disturbances tending towards hyperglycemia.

17. The insulin delivery controller of claim 15, wherein:
generating the insulin command signal for an insulin delivery unit includes generating the insulin command signal so that it will adjust the insulin delivery dosage amount and/or the insulin delivery dosage rate to modulate insulin delivery, wherein insulin delivery is modulated to:
approach a target glucose level.

18. A method for managing a processor configuration to efficiently attain an insulin delivery target, the method comprising:
processing glucose data including a data representation of glycemic disturbance (d(t));
determining a glucose rate of change (G′(t));
generating a command signal to dynamically reshape a glycemic disturbance within a prediction horizon of an insulin delivery controller according to the G′(t); and
generating an insulin command signal for an insulin delivery unit to adjust an insulin delivery dosage amount and/or an insulin delivery dosage rate.

19. The method of claim 18, wherein:
processing the glucose data, determining the (G′(t)), and generating the command signal to dynamically reshape the glycemic disturbance are performed via a closed loop control (CLC) process.

20. The method of claim 19, wherein:
the CLC process includes:
a model predictive control (MPC) algorithm;
a proportional integral derivative (PID) algorithm; or
a fuzzy logic (FL) algorithm.
